(19) United States
(12) Patent Application Publication
(10) Pub. No.: US 2010/0291246 A1
(43) Pub. Date: Nov. 18, 2010

(54) Anti-infection P-compound™

(76) Inventor: John T. Brady, Bronxville, NY (US)

Correspondence Address: Limin Wen, One Bungtown Road, Nichols Building, 1st Floor, Cold Spring Harbor, NY, 11724, US

(21) Appl. No.: 12/732,910

(22) Filed: Mar. 26, 2010

Related U.S. Application Data
(60) Provisional application No. 61/163,960, filed on Mar. 27, 2009.

(51) Int. Cl. A61K 36/82; A61P 31/12; A61P 31/04

(52) U.S. Cl. 424/729

(57) ABSTRACT

The present invention is related to a botanical extract, called P Compound, comprises anti-infection activities. The botanical sources, extracting process, formulation, and applications are disclosed within this invention.

### REFERENCE TO RELATED APPLICATION

This application claims the benefit of the filing date of U.S. Provisional Application Ser. No. 61/163,960, entitled “Anti-infection P-compound™” and filed on Mar. 27, 2009. The teachings of the entire referenced application are incorporated herein by references.

### FIELD OF THE INVENTION

This invention relates generally to natural compounds called “P Compound™” extracted from a botanical base source and methods of applying such compounds for topical external applications to eliminate infectious microbes on exposed surfaces, on humans and on livestock and reliably inhibit re-growth of bacteria, virus, fungus, parasite, and the like.

### BACKGROUND OF THE INVENTION

According to a science report from Science Daily (Dec. 6, 2007), hospitalizations related to a potentially deadly, antibiotic-resistant “super bug” more than doubled between 1999 and 2005, soaring from 127,000 to nearly 280,000. Mutated bacteria and virus microorganisms are sources of rapidly mounting serious infection throughout the world. These so called Super Bugs are prolific, stubbornly antibiotic resistant, highly contagious and a major concern for public health officials.

Antibiotic-resistant infections also impose greater costs. Several studies have estimated that antibiotic-resistant infections increase direct costs by 30 percent to 100 percent. MRSA-specific studies suggest that the additional cost of treating an antibiotic-resistant staph infection versus an antibiotic-sensitive infection range from a minimum of $3,000 to more than $35,000 per case. This suggests that such infections cost the health care system an extra $830 million to $9.7 billion in 2005, even without taking into account indirect costs related to patient pain, illness, and time spent in the hospital.

Staph, or Staphylococcus aureus, is a kind of bacteria that infect wounds and cause life-threatening infections, such as blood poisoning and pneumonia. The spread of staph has been formally grouped into two primary subsets: Health Care-Acquired infection in a hospital, clinic or dental care facility and Community-Acquired infection spreading to public venues such as schools.

One such super bug is called “MRSA” methicillin-resistant Staphylococcus aureus (MRSA), which is becoming prevalent in the broader community and already causing thousands of deaths each year in the United States.

MRSA belongs to a genus of bacteria—more than 30 types—called Staphylococcus, or just “staph.” Staphylococci are found in about 30% of adults, mostly on the skin or in the nose. Most people who carry the bug are healthy; they are said to be “colonized” but not infected. Most infections occur when one type of staph—Staphylococcus aureus—gets into the body through a break in the skin, such as a cut or burn. Even then, it usually causes only minor symptoms, such as small pimples or boils that can be treated without antibiotics. But staph infection can be very serious for people whose immune systems are weakened by age, illness, or chemotherapy. They may develop a deep abscess or life-threatening infection in the bloodstream, urinary tract, lungs, or surgical wounds.

Most serious staph infections can be successfully treated with methicillin, which belongs to the penicillin class of antibiotics. MRSA infections are resistant to this group of antibiotics, but they usually do respond to other, more potent antibiotics, particularly vancomycin. The trouble is that it may take a day or two to determine whether a given infection is caused by ordinary staph or MRSA. Because of the delay, patients infected with MRSA have longer hospital stays and worse outcomes, including higher death rates, than those with staph infections that are not resistant to methicillin.

MRSA are “super bugs” that have evolved resistance to most commonly used antibiotics, so they are more difficult and expensive to treat. MRSA and staph infections are now epidemic in many U.S. hospitals, long-term care facilities and communities. In all likelihood, MRSA infections are spreading in both hospitals and communities, complicating efforts to prevent infections in hospital patients. Hospital-acquired infections from all causes result in an estimated 90,000 deaths per year and are the sixth-leading cause of death nationally. They also increase patient suffering and the length of time patients spend in the hospital—in addition to direct health care costs, estimated to be more than $6 billion annually.

The National Centers for Disease Control and Prevention's Top 20 list ranks staph bacteria/MRSA #3 ahead of Autism, Pandemic Flu and Heart Disease (www.cdc.gov.). Traditional antiseptic/disinfectant products used by health care institutions and available to the public through retail outlets are unable to control or eliminate mobile ever-present Super Bugs.

Highly resistant mutant microorganisms are a persistent presence at the alarming level in all health care centers. Statistically, it has reached epidemic proportions with a ten-fold increase in debilitating, too often lethal, infection. Causes are invasive procedures on patients such as intravenous blood stream, organ surgery, catheters, skin punctures and through body openings.

Widespread use of lifesaving antibiotic drugs has led to the rise of mutated drug-resistant bacteria, or “super bugs,” which are outwitting even our newest and most powerful antibiotics. There is an emergency need for any natural compound (not antibiotics), which could eliminate these “super bugs”.

The present invention provides P Compound entirely extracted from natural plant source and the methods of using the same for an anti-infection purpose.

### SUMMARY OF THE INVENTION

The present invention is related to anti-infection P Compound that are extracted from a combination of Camellia Sinensis, Yerbe Mate, and Rooibos that are botanically sourced natural substances and such P Compound are used as proprietary formula topical medicines.

Topical P Compound in 100% natural formula, as a solo ingredient or primary ingredient additive, prevents infection by eliminating the presence of anti-biotic resistant bacteria and virus. Such topical antiseptic applications, without adding any extra chemical/anti-biotic supplement ingredients, effectively satisfy the needs of anti-infection natural solution.

Such botanical P Compound was extracted from a natural plant base source when combined with a liquid solution, which creates a formula for a topical antiseptic application. The method of applying P Compound eliminates the presence and re-growth of bacteria and virus types including MRSA that are harmful to humans and livestock. For example, P Compound completely destroyed the gram-positive bacteria (including Staphylococcus aureus, Bacillus cereus, Micrococcus luteus, Enterococcus faecium) and gram-negative bacteria (including Salmonella enteritides, Proteus vulgaris, Serratia marcescens, Psedumonas aeruginosa, Enterobacter aerogenes, Escherichia coli, Chromobacterium violaceum).

One topical application of the P Compound on human skin and livestock or when applied to exposed surfaces reliably killed staph/MRSA et al antibiotic resistant bacteria and eliminated immediate return and colony re-growth.

A lab test series confirmed one application of P Compound formula killed and continues to protect the treated area from the re-establishment of staph bacteria colonies for extended periods. In contrast, the generic ethyl alcohol based antiseptic products, due to rapid evaporation rates, required frequent applications and only resulted partial protection and did not eliminate most strains of highly resistant bacteria (Super bugs).

Topical P Compound, as an additive or a line extension product in healthcare industry, are natural ingredient compound replacements for chemicals/antibiotics. Topical P Compound have broad applications for use with a variety of institutional and consumer product categories where the elimination of staph type bacteria/virus presence is critical. Such natural anti-infection products will benefit to generic unregulated topical products in the retail medical/dental sector such as antiseptic wipes/sprays/liquids/soaps and reconstitute polluting/ineffective antibacterial soaps containing triclosan/triclocarban active ingredients.

This invented P Compound provides an alternative solution necessary to further control mutant forms of highly resistant microorganisms simultaneously addresses the concerns of every U.S. health care institution. These organizations are plagued by embarrassment, lawsuits and serious obstacles to professional healing standards. The credibility of these institutions would benefit greatly from products that effectively control, treat and eliminate the threat of Super Bug infection.

The development of natural solutions based on P Compound offers The following advantages: longer term treatment stability, infection prevention control, and environment green/positive alternatives.

P Compound ingredient additives upgrade existing antiseptic/disinfectant sector brands and foster expansion of new products with featured active ingredients in a number of institutional and consumer retail segments. When P Compound topical formulae are used as an additive ingredient mixed directly with the contents of an ethyl alcohol based antiseptic product it upgrades the efficacy of the product by eliminating populated colonies of Staph aureus MRSA, Strap mutants, and E faecalis bacteria. Similar experiments achieved the same result on Vancomycin resistant Enterococcus (VRE), Salmonella strains and Bacteriophage T1, which infects Escherichia coli B, was also adversely affected.

In summary, the present invention provides a natural, safe and effective solution to deal with microorganism infection including Super Bugs such as MRSA. The unique P Compound formula invented within this invention makes P Compound suitable to apply alone or mix with other existing antiseptic products as ingredient additives.

### BRIEF DESCRIPTION OF THE DRAWINGS

FIG. 1 Laboratory test screen of P Compound, as additives to Purell 8 oz. size, killed Super bug Staph aureus MRSA in bacteria control colonies.

FIG. 2 Laboratory test screen series of P Compound, as sole active ingredients, killed Staph aureus MRSA Super Bugs in bacteria control colonies.

FIG. 3 Laboratory test screen series of P Compound, as sole active ingredients, killed Bacillus cereus Super Bugs in bacteria control colonies.

### DETAILED DESCRIPTION OF THE PREFERRED EMBODIMENTS

P COMPOUND EXTRACT SOURCE

| | |
|---|---|
| BOTANICAL NAMES: | |
| TEA | Camellia Sinensis, Unfermented |
| ROOIBOS | Aspalathus linearis |
| YERBA MATE (MATE) | Ilex Paraguayensis |
| APPEARANCE: | Brown Powder |
| TASTE: | Bitter/Astringent |
| WATER SOLUBILITY: | Soluble |
| COMMON NAME: | Tea Extract |
| POLYPHENOL CONTENT: | Minimum 74% |
| CATECHIN CONTENT: | Minimum 50% |
| EGCG CONTENT: | Minimum 24% |
| MICROBIOLOGICAL DATA: | |
| YEAST | Max 100 cfu/gram |
| MOLD | Max 100 cfu/gram |
| TOTAL PLATE COUNT | Max 1000 cfu/gram |
| E. Coli | Negative |
| STORAGE: | Ambient temperature; avoid moisture. |

### P Compound Extraction Process:

Raw materials from Camellia Sinensis, Rooibos, and Yerbe Mate were suspended in hot water (80° C.), P Compound were extracted according the following steps: Extraction - - - Filtration - - - Cooling - - - Concentration - - - Cooling - - - Extracted by Ethyl Acetate - - - Concentrating the liquor with Centrifugation - - - Spraying and drying - - - Sifting and blending

### P Compound Anti-Infection Effects:

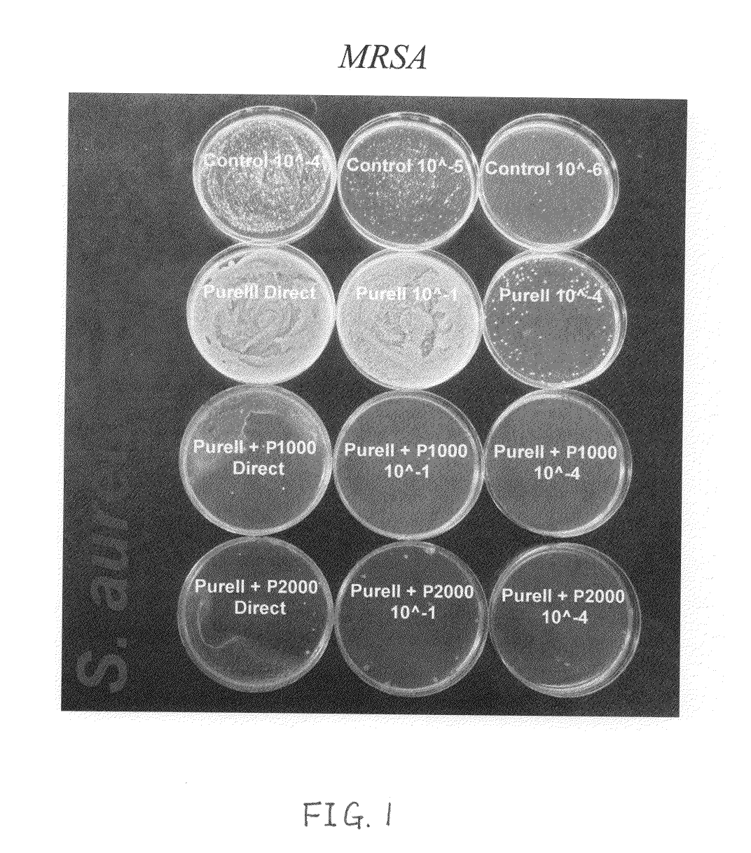

Clinical product testing was performed under the auspices of an independent microbiological laboratory(s). The test objective was to determine the feasibility/compatibility of a P (Prevent) Compound additive to the ingredients present in a well-established antiseptic brand. A quantity of P Compound formula, directly added to the leading antiseptic (ethyl alcohol based) packaged brand, produced unprecedented results. When P Compound direct additive ingredient was included, one topical application reliably and totally eliminated populated colonies of Staph aureus MRSA, Strap mutants and E faecalis bacteria. Conversely, absent the P Compound additive, topical pre-testing of this leading brand has determined repeated applications were unable to eliminate colonies comprised of the same drug resistant bacteria. See FIG. 1 for comparative product test screen results (P formula screens illustrating the results of diluted and undiluted bacteria control zone of inhibition experiments). Also, a two-year series of clinical microbiological studies has been conducted. The result documents unprecedented success of P compound in reliably eliminating the presence of Staph aureus, Strap mutants, E faecalis, MRSA bacteria and BacteriophageT 1.

Other leading national antiseptic brands under testing are unable to claim elimination of drug resistant Super Bugs such as Staph/MRSA bacteria/human viruses.

In addition, early research trials with P Compound has demonstrated similar results in eliminating Vancomycin-Resistant Enterococcus (VRE), a new mutant strain of deadly Enterococcus bacteria for which there is no antibiotic drug cure. Typically, all the above referenced microorganisms begin as health care acquired infections with hospital/dental personnel as the initial carriers.

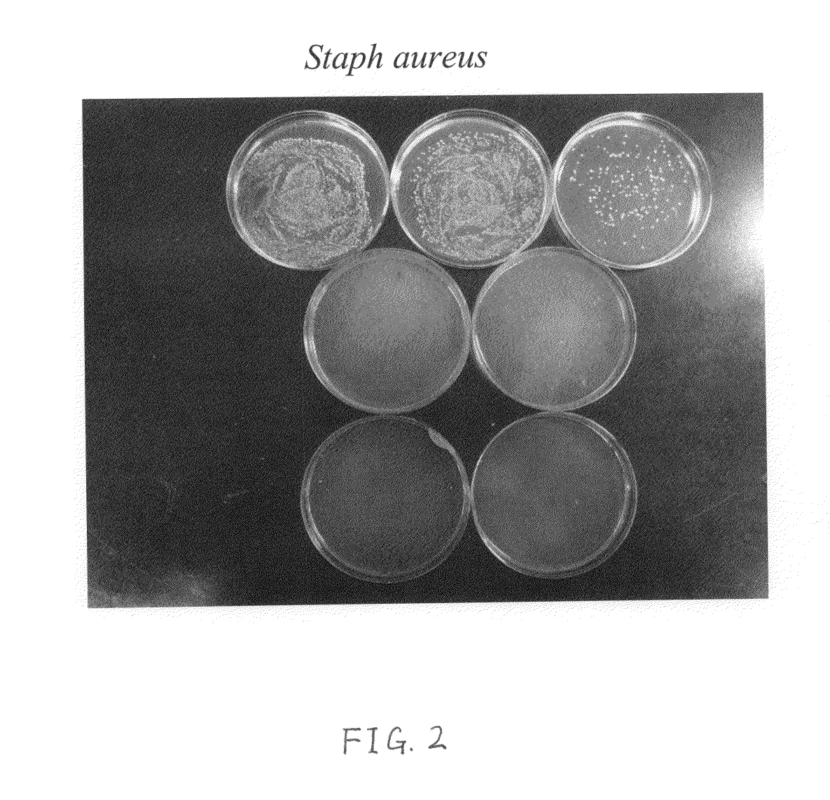

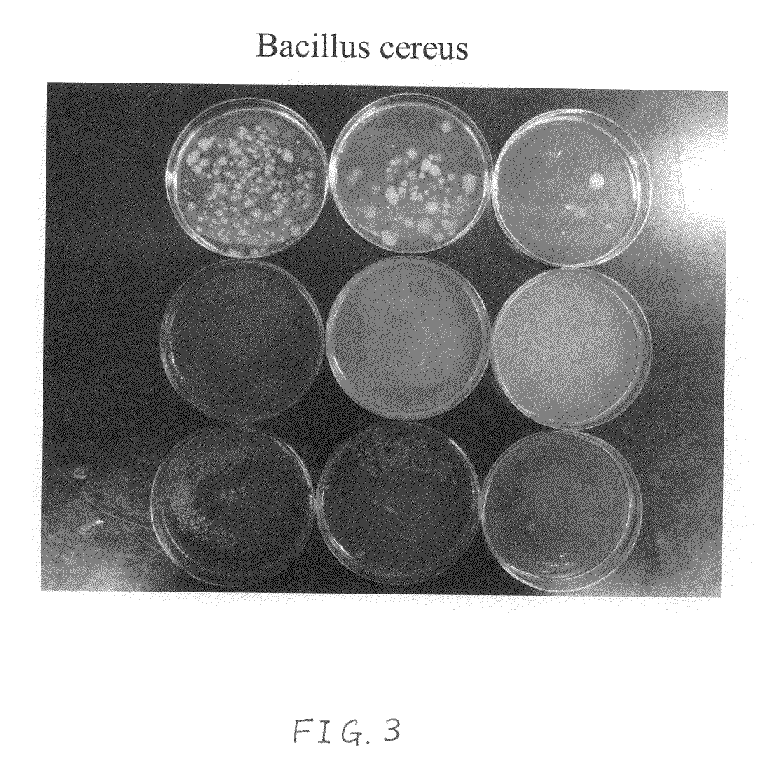

For anti-bacteria testing, the antibacterial effect of P Compound on S. aureus and B. cereus was obtained by observing zones of inhibition of bacteria grown on Mannitol Salt agar (Kirby-Bauer technique). The zone of inhibition was equally pronounced for P compound and is significant in that we are unaware of other topically applied products, commercially available, offering this level of a solution. A series of photographs in support of these findings, based on diluted and undiluted bacteria control experiments, are enclosed herewith to serve as documentation of these results (See FIG. 2 and FIG. 3).

Our results showed that the antibacterial effect of P Compound was significant. For example, 1% of P Compound dissolved in water totally destroyed Staphylococcus aureus; 6% of P Compound dissolved in water totally destroyed Salmonella enteritides. These natural extract compounds had a substantially greater effect on the gram positives than on the gram negatives. The antibacterial effects of these additives were obtained by observing zones of inhibition of bacteria grown on either Mueller Hinton II agar or Hektoen Enteric agar (Kirby-Bauer technique), and by direct plate counts of bacterial colonies grown on Tryptic Soy agar using the standard plate technique. The antibacterial effect of an instant hand sanitizer was also investigated. The addition of P Compound to an instant hand sanitizer destroyed Staphylococcus aureus and Bacillus cereus. In absence of the additives, the hand sanitizer was ineffective.

The following table 1 showed that the anti-MRSA effects of 7.5% of P Compound.

TABLE 1
Anti-MRSA effects of P Compound

| | | Test | | | | |
|---|---|---|---|---|---|---|
| | | Population | | | | |
| | | Control | Number of | Log₁₀ | | |
| | Exposure | CFU/mL* | Survivors | Number of | Percent | Log₁₀ |
| Test Organism | Time | (Log₁₀) | (CFU/mL)* | Survivors | Reduction | Reduction |
| Methicillin | 1 minute | 5.6 × 10⁵ | 3.4 × 10⁵ | 5.53 | 39.3% | 0.22 |
| Resistant | | (5.75) | | | | |
| Staphylococcus | 1 hour | 4.5 × 10⁵ | <5 | <0.7 | >99.99% | >4.95 |
| aureus - | | (5.65) | | | | |
| MRSA (ATCC | 6 hours | 6.3 × 10⁵ | <5 | <0.7 | >99.99% | >5.1 |
| 33592) | | (5.80) | | | | |
| | 24 hours | 3.3 × 10⁵ | <5 | <0.7 | >99.99% | >4.82 |
| | | (5.52) | | | | |

*Colony forming units per mL of test mixture

The following P Compound Test Results also showed that 1 ml of different concentrations of P Compound (0.2%; 2%; or 10%) with different exposing times to control MRSA bacteria could eliminate the growth or colony forming of bacteria significantly.

Method 1. 1 ml of different concentrations of P Compound (0.2%; 2%; or 10%) was added to a TSA agar plate and then added 0.1 ml of MRSA bacterial organism with different dilutions and spread. Plates are placed in a 37° C. incubator and examined colonies of bacteria after 18-24 hours.

Result from Method 1.
MRSA Bacterial concentration = 21 × 10⁸/ml

| | Dilutions | | | |
|---|---|---|---|---|
| | 10⁻² | 10⁻⁴ | 10⁻⁶ | 10⁻⁷ |
| Control | Too much to | Too much to count | 210 colonies | 21 colonies |
| | count | | | |
| 0.2% | No Colony | No Colony | No Colony | No Colony |
| 2.0% | No Colony | No Colony | No Colony | No Colony |
| 10.0% | No Colony | No Colony | No Colony | No Colony |

Note:
No Colony means total elimination of bacteria.

Method 2. 1 ml of different concentrations of P Compound (0.2%; 2%; or 10%) was added to a TSA agar plate and then added 0.1 ml of MRSA bacterial organism with different dilutions and spread. Plates are placed in a 37° C. incubator and examined colonies of bacteria after 30 seconds and 15 minutes.

Result from Method 2.
MRSA Bacterial concentration = 29 × 10⁸/ml

| | Dilutions | | | |
|---|---|---|---|---|
| | 10⁻² | 10⁻⁴ | 10⁻⁶ | 10⁻⁷ |
| Control | Too much to count | Too much | 290 colonies | 29 colonies |
| | | to count | | |

30 seconds exposure

| | | | | |
|---|---|---|---|---|
| 0.2% | Too much to count | Too much | 38 colonies | No Colony |
| | | to count | | |
| 2.0% | Some Colony | No Colony | No Colony | No Colony |
| 10.0% | No Colony | No Colony | No Colony | No Colony |

15 minutes exposure

| | | | | |
|---|---|---|---|---|
| 0.2% | Too much to count | Too much | 29 colonies | No Colony |
| | | to count | | |
| 2.0% | No Colony | No Colony | No Colony | No Colony |
| 10.0% | No Colony | No Colony | No Colony | No Colony |

Note:
No Colony means total elimination of bacteria.

For anti-virus testing, Bacteriophage T1, which infects Escherichia coli B, was adversely affected by P Compound. The percent loss of titer, due to the addition of P Compound, was observed for the phage after ten minutes of intermittent mixing at room temperature. After one hour (without mixing), T1 was totally inactivated.

Further research is also warranted in testing P Compound as a nutraceutical or as a raw material that may have widespread implications in the treatment of potentially pathogenic microorganisms.

All ingredients contained in P Compound are naturally derived and generally recognized as safe based on the Food & Drug Administration and FEMA GRAS LISTS. P Compound ingredients can be added into hand sanitizers, soaps, baby wipes, moist towelettes, etc.

Although the invented P Compound and the method of using same according to the present invention has been described in the foregoing specification with considerable details, it is to be understood that modifications may be made to the invention which do not exceed the scope of the invention claims and modified forms of the present invention done by others skilled in the art to which the invention pertains will be considered infringements of this invention when those modified forms fall within the claimed scope of this invention.

What is claimed is:

1. A botanical extract from a combination of natural Camellia Sinensis, Yerbe Mate, and Rooibos comprises anti-infection activities, wherein said botanical extract named P Compound when added to distilled water in solution is used as proprietary formula topical medicines.

2. The P Compound mentioned in claim 1, wherein the P Compound is extracted according to the following steps after suspended in 80° C. water: extraction, filtration, cooling, concentration, cooling, extracted by Ethyl Acetate, concentrating the liquor through centrifugation, spraying and drying, sifting and blending.

3. The P Compound mentioned in claim 1, wherein a formula of said P Compound comprises 0.02%-10% of said P Compound by weight in distilled water solution.

4. The P Compound said in claim 1 comprises antibacterial effect, wherein the P Compound eliminates and/or prevents re-growth of gram-positive and gram-negative bacteria including but not limited to antibiotic-resistant bacteria.

5. The antibiotic-resistant bacteria said in claim 4 include but not limited to methicillin-resistant Staphylococcus aureus and vancomycin-resistant Enterococcus.

6. The P Compound said in claim 1 comprises anti-virus effect, wherein the P Compound eliminates and/or prevents re-growth of Bacteriophage T1.

7. A method to directly apply the P Compound said in claim 1 to a surface in the treatment of potential pathogenic microorganisms, wherein the P Compound is suitable to apply alone or mix with other existing antiseptic products as ingredient additives.
